(19) United States
(12) Patent Application Publication
(10) Pub. No.: US 2024/0122897 A1
(43) Pub. Date: Apr. 18, 2024

(54) DMT SALTS AND THEIR USE TO TREAT BRAIN INJURY

(71) Applicant: ALGERNON PHARMACEUTICALS INC., Vancouver (CA)

(72) Inventors: Mark WILLIAMS, Winnipeg (CA); Christopher BRYAN, Vancouver (CA)

(21) Appl. No.: 18/274,896

(22) PCT Filed: Jan. 28, 2022
(86) PCT No.: PCT/CA2022/050121

§ 371 Date: Jul. 28, 2023

Related U.S. Application Data
(60) Provisional application No. 63/143,688, filed on Jan. 29, 2021.
(60) Provisional application No. 63/273,612, filed on Oct. 29, 2021.
(60) Provisional application No. 63/187,681, filed on May 12, 2021.
(60) Provisional application No. 63/143,679, filed on Jan. 29, 2021.
(60) Provisional application No. 63/143,695, filed on Jan. 29, 2021.

(51) Int. Cl. A61K 31/4045; A61P 9/10; A61P 25/16

(52) U.S. Cl. CPC A61K 31/4045; A61P 9/10; A61P 25/16

(57) ABSTRACT

DMT nicotinate and DMT pamoate are disclosed. DMT and its salts are found useful in the treatment of stroke, multiple sclerosis, Parkinson's disease, and traumatic brain injury. They are particularly useful in the treatment of haemorrhagic stroke. They are particularly useful in the immediate treatment of undiagnosed stroke. They are for use with anti-hypertensives when treating haemorrhagic stroke or TBI. Also taught are preferred dosages and serum levels of DMT, an intravenous pump device containing a pharmaceutically acceptable form of DMT. The pump may be configured to provide a specific dose and/or duration. The pump may have a locking system to prevent access to said compound or said DMT above the dose.

### FIELD OF INVENTION

The invention pertains to DMT salt compositions. The invention pertains to DMT to treat brain injury such as stroke, multiple sclerosis, Parkinson's disease, and traumatic brain injury, including haemorrhagic stroke. The invention pertains to DMT formulations, dosages, methods and devices

### CROSS REFERENCE TO RELATED APPLICATIONS

The present application claims priority to US provisional application No. 63/143,679, filed Jan. 29, 2021, U.S. provisional application No. 63/143,688, filed Jan. 29, 2021, U.S. provisional application No. 63/143,695, filed Jan. 29, 2021, U.S. provisional application No. 63/187,681, filed May 12, 2021 and U.S. provisional application No. 63/273,612, filed Oct. 29, 2021.

### BACKGROUND

Each year there are approximately 15M strokes that occur globally with 700,000 strokes occurring in the U.S. alone. Approximately 85% of all strokes are ischemic strokes which occur when a blood clot blocks blood flow to the brain.

Currently, medication treatment for ischemic stroke are primarily limited to Tissue Plasminogen Activator (“tPA”) or blood thinners. However, these treatments are stroke type specific and cannot be given until the patient has been provided a CT scan to determine if the stroke is ischemic or haemorrhagic. Patients being treated with tPA must receive the drug within 4.5 hours of the injury. As a result, only 5% of stroke patients receive tPA.

A haemorrhagic stroke is due to bleeding in or around the brain. It as also referred to as a brain haemorrhage or a brain bleed and occurs in approximately 15% of all stroke cases. Administration of tPA or blood thinners to a patient suffering haemorrhagic stroke would be extremely detrimental. Thus the type of stroke (haemorrhagic versus ischemic) must be diagnosed before any medication can be administered.

Multiple sclerosis (MS) is a condition that can affect the brain and spinal cord, causing a wide range of potential symptoms, including problems with vision, arm or leg movement, sensation or balance. It's a lifelong condition that can sometimes cause serious disability, although it can occasionally be mild.

Parkinson's disease is a brain disorder that leads to shaking, stiffness, and difficulty with walking, balance, and coordination. Parkinson's symptoms usually begin gradually and get worse over time. As the disease progresses, people may have difficulty walking and talking.

Traumatic Brain Injury (TBI) is a disruption in the normal function of the brain that can be caused by a blow, bump or jolt to the head, the head suddenly and violently hitting an object or when an object pierces the skull and enters brain tissue. The following clinical signs constitutes alteration in the normal brain function: loss of or decreased consciousness; loss of memory for events before or after the event (amnesia); focal neurological deficits such as muscle weakness, loss of vision, change in speech; alteration in mental state such as disorientation, slow thinking or difficulty concentrating.

Symptoms of a traumatic brain injury can be mild, moderate, or severe, depending on the extent of damage to the brain. Mild cases may result in a brief change in mental state or consciousness. Severe cases may result in extended periods of unconsciousness, coma, or even death.

DMT, or N, N-Dimethyltryptamine is a hallucinogenic tryptamine drug producing effects similar to those of other psychedelics like LSD, psilocybin and psilocin. DMT occurs naturally in many plant species and animals and has been used in religious ceremonies as a traditional spiritual medicine. DMT can also be synthesised in laboratory.

DMT is believed to activate pathways involved with forming neuron connections and has been shown to increase the number of dendritic spines on cortical neurons. Dendritic spines form synapses (connections) with other neurons and are a major site of molecular activity in the brain.

Data from a study published in Experimental Neurology, in May 2020 showed that in a rat model of cerebral ischemia-reperfusion injury, DMT reduced the infarct (dead cells) volume as well as improved functional recovery. Although it has been reported that DMT is useful in the treatment of ischemic stroke in rat models, there was no evidence that DMT is useful in the treatment of hemorrhagic stroke. Known treatments for ischemic stroke are dangerous if applied to hemorrhagic stroke.

### SUMMARY OF INVENTION

The invention teaches salts of DMT, namely DMT nicotinate and DMT pamoate.

The invention also teaches a method of treating neuronal injury, including, in preferred embodiments, stroke, multiple sclerosis, Parkinson's disease, and traumatic brain injury, comprising administering salts of DMT.

The invention also teaches a method of treating haemorrhagic stroke, multiple sclerosis, Parkinson's disease, and traumatic brain injury, comprising administering salts of DMT.

In another embodiment, the invention teaches a pharmaceutically acceptable form of DMT for the treatment of stroke, multiple sclerosis, Parkinson's disease, and traumatic brain injury, comprising administration in combination with, and preferably before, constrained exercise. The administration can be 2-4 days before constrained exercise. Preferably at least 2 days before constrained exercise. Preferably about 3 days before constrained exercise. The constrained exercise comprises physical exercise during which the healthy side of a patient's body is constrained.

In another embodiment the invention teaches the use of a pharmaceutically acceptable form of DMT for the treatment of stroke, multiple sclerosis, Parkinson's disease, and traumatic brain injury, comprising administration commencing prior to a diagnosis of ischemic or haemorrhagic stroke. In the uses and methods of the present invention, the stroke can be haemorrhagic stroke.

The administration of DMT may be at a rate of about 0.001 to 50 mg DMT/kg patient bodyweight/hour. Preferably, administration is at a rate to of about 0.005 to 20 mg/kg/hour. Preferably, administration at a rate of about 0.01 to 5 mg/kg/hour. Preferably administration at a rate of about 0.5 mg/kg/hour. The administration of DMT can be at a rate to provide a serum level of about 0.05 to 250 ng/ml Preferably at a rate to provide a serum level of about 0.1 to 150 ng/ml. More preferably at a rate to provide a serum level of about 1.0 to 50 ng/ml. Preferably at a rate to provide a serum level of about 25 ng/ml. In a preferred clinical protocol, an IV bolus dose is administered which precedes the infusion. The preferred level of the bolus is 0.005 to 0.4 mg/kg, preferably 0.01 to 0.2 mg/kg, preferably about 0.1 mg/kg.

The administration of DMT can be for a duration of about 15 minutes to 24 hours. Preferably for a duration of about 1 hour to 18 hours. Preferably for a duration of about 2 hours to 12 hours. Preferably administration for a duration of about 6 hours.

In another embodiment, the method or use of a pharmaceutically acceptable form of DMT further comprises administration with an antihypertensive for the treatment of stroke or TBI.

In a preferred embodiment, in the methods or uses recited herein utilize pamoate DMT or nicotinate DMT.

In another embodiment, the invention teaches a device comprising an intravenous pump, said device containing a pharmaceutically acceptable form of DMT. In a preferred embodiment, the pump is configured to provide the doses set out herein. In a preferred embodiment the pump is configured to provide the dose for the durations set out herein.

In embodiments, the device further comprises a locking system to prevent access to said DMT above the doses recited herein. The locking system may lock a container of the compound. The locking system may lock or control adjustment of the rate of administration of the compound.

### BRIEF DESCRIPTION OF FIGURES

FIG. 1 is a bar graph showing mean total length of processes and branches per cell.

FIG. 2 is a bar graph showing mean total length of processes per cell.

FIG. 3 is a bar graph showing mean total length of branches per cell.

FIG. 4 is a bar graph showing mean number of processes and branches per cell.

FIG. 5 is a bar graph showing mean number of processes per cell.

FIG. 6 is a bar graph showing mean number of branches per cell.

FIG. 7 is a bar graph showing mean longest branch length per cell.

FIG. 8 is a line graph showing DMT fumarate's binding activity to 5HT2A receptor against a standard.

FIG. 9 is a line graph showing DMT fumarate's binding activity to sigma-1 receptor against a standard.

FIG. 10 is a line graph showing DMT nicotinate's binding activity to 5HT2A receptor against a standard.

FIG. 11 is a line graph showing DMT pamoate's binding activity to 5HT2A receptor against a standard.

FIG. 12 is a line graph showing DMT pamoate's binding activity to sigma-1 receptor against a standard.

### DETAILED DESCRIPTION

The inventors have found new and useful salts of DMT, namely DMT nicotinate and DMT pamoate.

The present inventors have found that DMT nicotinate displayed clear binding potency and good affinity to the receptors 5HT2a and sigma-1. The present inventors have further found that DMT pamoate displayed clear binding potency and an improvement in 5HT2a and sigma-1 affinity at higher concentrations than other forms of DMT discussed herein. Using the evidence provided herein and as these receptors are highly correlated with neuronal activity, and in particular with DMT activity in the brain, these DMT salts can provide improvements over known salts of DMT.

The invention also teaches a method of treating neuronal injury, including, in preferred embodiments, stroke, multiple sclerosis, Parkinson's disease, and traumatic brain injury, comprising administering salts of DMT.

The inventors have found that DMT can have a positive effect in the treatment of neuronal damage, such as by ischemic stroke. DMT can have positive or at least neutral effect in the treatment of hemorrhagic stroke. This is an important finding for treatment or prevention of strokes. Drugs that worsen hemorrhagic stoke, such as tissue plasminogen activator, must be delayed until the type of stroke that a patient is suffering is determined. It is important that treatment begin as soon as possible to improve patient outcomes. Therefore as DMT treatment has a positive or at least neutral effect whether the stroke is hemorrhagic or ischemic, it can be used as an immediate, first response in the treatment of stroke, even prior to a diagnosis as to the type of stroke.

As used herein, the terms “treatment” or “therapy” (as well as different word forms thereof) includes preventative (e.g., prophylactic), curative or palliative treatment.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects. As an example, the compounds useful in the methods of the present invention are administered at a dosage and for a time such that the level of activation and adhesion activity of platelets is reduced as compared to the level of activity before the start of treatment.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

The active compounds described above may be formulated for administration in a pharmaceutical carrier in accordance with known techniques. See, e.g., Remington, The Science and Practice of Pharmacy (9th Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising DMT or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

In addition to the active compounds, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

Throughout the description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

While a number of exemplary aspects and embodiments are discussed herein, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### Example 1—the Effects of DMT in a Rat Model of Stroke

Rats are deeply anesthetized with 3% isoflurane in an induction chamber. After loss of consciousness, the rats are fixed on the stereotactic frame using a nose clamp and two ear bars, and the temperature of rats is maintained at 37° C. by a temperature controller. The rats are kept anesthetized with 2% isoflurane by gas mask.

Using the stereotactic apparatus, a left point 3 mm lateral to the bregma, and 1 μl collagenase type IV (0.25 IU/μl) is injected into corpus striatum (5 mm below the skull) by a 5 μl Hamilton syringe 26 G at a slow rate of 0.2 μl/min. The syringe is remained at the place about 7 min after the injection is completed. For about 7 min later, the syringe is removed slowly

Once the syringe is removed, sterile bone wax is used to plug the hole quickly. The rats are removed from the stereotactic apparatus and are allowed to recover in a warmed cage with free access to food and water.

Treatment starts immediately before the removal of the filament by an intra-peritoneal injection of a bolus composed of 1 mg/Kg bw N,N-dimethyl-tryptamine (DMT) dissolved in 0.1 ml 70% ethanol, diluted to 1 ml with saline a continuous infusion of DMT at 2 mg/Kg bw/h dose was delivered via intra-peritoneally placed osmotic pumps for 24 h.

To evaluate the rat Model of ICH induced by collagenase IV, the rats are sacrificed and the brain is cut into slices after operation to assess the volume of hematoma. Furthermore, the neurobehavior (Bederson test) of ICH rats is assessed and the volume of hematoma also is analysed by MRI or histologically.

### Example 2—Novel Salts of DMT

Various acids, including oleic acid, benzoic acid, fumaric acid, nicotinic and pamoic acid improve stroke outcomes (Song J, Kim Y S, Lee D H, et al. Neuroprotective effects of oleic acid in rodent models of cerebral ischaemia. Sci Rep. 2019; 9(1); Sharmin O, Abir A H, Potol A, et al. Activation of GPR35 protects against cerebral ischemia by recruiting monocyte-derived macrophages. Sci Rep. 2020; 10(1):1-13; Nardai S, Laszlo M, Szabo A, et al. N,N-dimethyltryptamine reduces infarct size and improves functional recovery following transient focal brain ischemia in rats. Exp Neurol. 2020; 327:113245).

DMT Fumarate is the only form of DMT approved for research by the FDA.

The fumarate salt of DMT has been consistently used as other salts (e.g., acetate, citrate, hydrochloride, etc.) tend to be hygroscopic (Cameron and Olson, Dark Classics in Chemical Neuroscience: N,N-Dimethyltryptamine, ACS Chem Neuroscience, October 17; 9(10):2344-2357, 2018). A survey of the literature (Wikipedia, Tihkal (https://www.erowid.org/li brary/books_online/tihkal/tihkal06.shtml), show that DMT salts predominantly either do not produce crystal forms (“waxes” is the most common description), or produce solid forms which are hygroscopic or even deliquescent. Extremely hygroscopic compounds will not readily crystallize.

DMT is not readily water soluble and the salt form is preferred for preparation of solutions. The inventors determined whether it was possible to produce particular salts with particular counter ions. Experiments were necessary to determine whether or not various salts of DMT were possible. If the salt could be successfully made, its biophysical properties were accessed to confirm that it is acceptable for parenteral administration.

Unfortunately, many possible types of DMT salts did not provide the ease of synthesis, improved properties etc. that are needed for pharmaceuticals. In some cases, the physical nature of the salt made it impractical to test the compound in a biological assay. Some salts of DMT, such as oleic DMT, are pharmaceutically unusable. For example, oleate DMT when prepared produced a sticky oil, rather than a solid product.

### Example 2A—Nicotinate (Niacin) Salts of DMT

Administration of nicotinic acid improves stroke outcomes. The nicotinate DMT salt can have improved physicochemical properties.

Nicotinate DMT may improve the core drug in a number of ways: improved absorption (from improved solubility and dissolution), altered release profile (i.e. extended release properties), improved temperature stability, improved photostability (i.e. less likely to break down with light exposure), improved stability to moisture (i.e. improved hygroscopicity), improved palatability (i.e. improved taste), improved efficacy (some salt forms elicit different effects on the body, altered melting point (useful in drug manufacturing), improved compatibility (useful in drug manufacturing), improved pH of the parent drug compound (i.e. acidic or basic), improved solubility of the compound, improved safety/tolerability of the salt form, and reduced adverse effects physiologically.

To produce the nicotinate salt, DMT free base can be dissolved in a suitable organic solvent. For example, 5.00 g, 26.6 mmol dissolved in 100 mL acetone. The solvent is combined with a solution of nicotinate acid in a suitable organic solvent, combined with 350 mL of an acetone solution of acid (1.54 g, 13.28 mmol) in a 500 mL Erlenmeyer flask. After solutions are combined and cooled as a suspension stored at 4° C. as white crystals of nicotinate DMT salt crystals the salt is isolated by filtration, vacuum filtered using a Büchner funnel with Whatman #2 filter paper, washed with 2×75 mL cold acetone, then placed in the 40° C. vacuum oven and dried to constant weight.

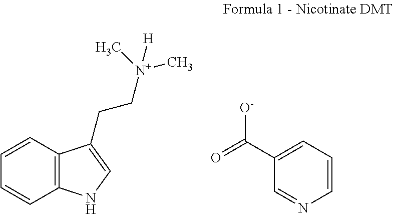

### Example 28—Pamoate Salts of DMT

Administration of pamoic acid improves stroke outcomes. The pamoic DMT salt can have improved physicochemical properties.

Pamoic DMT salt can improve the core drug in a number of ways: improved absorption (from improved solubility and dissolution), altered release profile (i.e. extended release properties), improved temperature stability, improved photostability (i.e. less likely to break down with light exposure), improved stability to moisture (i.e. improved hygroscopicity), improved palatability (i.e. improved taste), improved efficacy (some salt forms elicit different effects on the body, altered melting point (useful in drug manufacturing), improved compatibility (useful in drug manufacturing), improved pH of the parent drug compound (i.e. acidic or basic), improved solubility of the compound, improved safety/tolerability of the salt form, and reduced adverse effects physiologically.

To produce the pamoate salt, DMT free base can be dissolved in a suitable organic solvent. For example, 5.00 g, 26.6 mmol dissolved in 100 mL acetone. The solvent is combined with a solution of pamoate acid in a suitable organic solvent. Combined with 350 mL of an acetone solution of acid (1.54 g, 13.28 mmol) in a 500 mL Erlenmeyer flask. After solutions are combined and cooled as a suspension stored at 4° C. as white crystals of pamoate DMT salt crystals the salt is isolated by filtration, vacuum filtered using a Büchner funnel with Whatman #2 filter paper, washed with 2×75 mL cold acetone, then placed in the 40° C. vacuum oven and dried to constant weight.

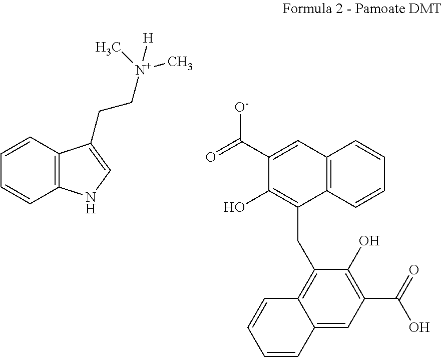 [SMILES: C[NH+](C)CCc1c[nH]c2ccccc12 ; O=C([O-])c1cc2ccccc2c(Cc2c(O)c(C(=O)O)cc3ccccc23)c1O]

### Example 3—Use of Pamoate or Nicotinate DMT for the Treatment of Neuronal Injury

The present inventors demonstrate that the usefulness of novel pamoate and nicotinate salt forms of DMT for the treatment of neuronal injury, such as by ischemic or hemorrhagic stroke, multiple sclerosis, Parkinson's disease, and traumatic brain injury. These salt forms of DMT may be preferred over other salt forms of DMT (e.g. hemi fumarate). These salts possess the appropriate biophysical characteristics for parenteral use. The present invention thus relates to salts of DMT and their use in parenteral injections.

Thus pamoate DMT is a preferred salt. DMT has therapeutic benefit in the treatment of stoke, multiple sclerosis, Parkinson's disease, and traumatic brain injury. Pamoic acid has therapeutic benefit in the treatment of stoke, multiple sclerosis, Parkinson's disease, and traumatic brain injury. DMT pamoate salt can have efficacy for the treatment of stoke compared to e.g. the fumarate salt.

Thus nicotinate DMT is also a preferred salt. DMT has therapeutic benefit in the treatment of stoke, multiple sclerosis, Parkinson's disease, and traumatic brain injury. Nicotinic acid has therapeutic benefit in the treatment of stoke, multiple sclerosis, Parkinson's disease, and traumatic brain injury. DMT nicotinate salt can have improved efficacy for the treatment of stoke compared to e.g. the fumarate salt.

For treatment of multiple sclerosis, Parkinson's disease, and traumatic brain injury, fumarate DMT is another useful salt.

### Example 4—the Effects of Nicotinate or Pamoate DMT in a Rat Model of Stroke

Rats are deeply anesthetized with 3% isoflurane in an induction chamber. After loss of consciousness, the rats are fixed on the stereotactic frame using a nose clamp and two ear bars, and the temperature of rats is maintained at 37° C. by a temperature controller. The rats are kept anesthetized with 2% isoflurane by gas mask.

Using the stereotactic apparatus, a left point 3 mm lateral to the bregma, and 1 μl collagenase type IV (0.25 IU/μl) is injected into corpus striatum (5 mm below the skull) by a 5 μl Hamilton syringe 26 G at a slow rate of 0.2 μl/min. The syringe is remained at the place about 7 min after the injection is completed. For about 7 min later, the syringe is removed slowly

Once the syringe is removed, sterile bone wax is used to plug the hole quickly. The rats are removed from the stereotactic apparatus and are allowed to recover in a warmed cage with free access to food and water.

Treatment starts immediately before the removal of the filament by an intra-peritoneal injection of a bolus composed of 1 mg/Kg bw N,N-dimethyl-tryptamine (DMT) nicotinate or pamoate salt dissolved in 0.1 ml 70% ethanol, diluted to 1 ml with saline a continuous infusion of nicotinate or pamoate DMT at 2 mg/Kg bw/h dose was delivered via intra-peritoneally placed osmotic pumps for 24 h.

To evaluate the rat Model of ICH induced by collagenase IV, the rats are sacrificed and the brain is cut into slices after operation to assess the volume of hematoma. Furthermore, the neurobehavior (Bederson test) of ICH rats is assessed and the volume of hematoma also is analysed by MRI or histologically.

### Example 5—the Effects of Nicotinate or Pamoate DMT in a Rat Model of Stroke

The transient MCAO (middle cerebral artery occlusion) model is applied on male Wistar rats under isoflurane anaesthesia. Following the surgical exposure of the right internal carotid artery, the suture is positioned while monitoring of the cerebral blood flow over the right middle cerebral artery territory with Laser-Doppler Flowmetry. Animals with a perfusion-drop of at least 40% are randomized for the treatment arms, the ischemia was maintained for 60 min.

Treatment started immediately before the removal of the filament by an intra-peritoneal injection of a bolus composed of 1 mg/Kg bw N,N-dimethyl-tryptamine (DMT) nicotinate or pamoate salt dissolved in 0.1 ml 70% ethanol, diluted to 1 ml with saline with the counter acid at the appropriate 1:1 or 1:2 stoichiometry. Afterwards a continuous infusion of nicotinate or pamoate DMT at 2 mg/Kg bw/h dose was delivered via intra-peritoneally placed osmotic pumps for 24 h. Rats in the control group received a vehicle bolus only, while a third group of animals received 1-(3,4-Dichlorophenethyl)-4-methylpiperazine dihydrochloride in parallel with the DMT via separate osmotic pumps at 2 mg/kg bw/24 h dose-rate, following a 1 mg/kg-body weight loading dose. Motor function and infarct volumes were measure as before or histologically for the latter.

### Example 6—the Effects of DMT Administration in a Rat Model of Traumatic Brain Injury (TBI)

The controlled cortical impact (CCI) model of traumatic brain injury is widely used to investigate the possible effects of protective or restorative treatments for structural damage and functional deficits caused by head injury or concussion (Charles River Disovery Services, Finland). The therapeutic effects of DMT treatment when administered at different times after the injury is shown.

Adult male CD rats (250-300 g) are housed under controlled temperatures (23° C.) in a 12-h light/dark cycles with access to food and water ad libitum. All procedures are in accordance with local regulations and institutional animal care guidelines. Animals are divided into 2 groups, each with 9 subgroups as described below.

### Design

Group 1: Sham surgery
Group 2: CCI+vehicle treatment @ 1 hour and 3 days after reperfusion
Group 3: CCI+DMT treatment (1 mg/kg) @ 1 hour and vehicle treatment @ 3 days after reperfusion
Group 4: CCI+vehicle treatment @ 1 hour and DMT treatment (1 mg/kg) @ 3 days after reperfusion
Group 5: CCI+DMT treatment @1 hour and DMT treatment @ 3 days after reperfusion (1 mg/kg)

All groups other than the sham group undergo CCI. Rats are anesthetized with isoflurane and a midline craniectomy is performed to expose the dura covering the medial frontal cortex. A calibrated impact is made on the midline medial frontal cortex. PinPoint™, a Precision Cortical Impactor™ is used for inducing traumatic brain and spinal cord injuries in a medical research model. The is system is designed to provide the user with precision control, power, and flexibility to generate accurate, reliable, and reproducible results.

Body temperature is maintained at 37° C. by a heating pad throughout the surgical procedure and vital signs are monitored until the animal recovers from surgery.

Vehicle (saline solution) or DMT are given as an IV bolus of (low dose or high dose) followed by an 1-hour intravenous infusion with concentrations and volumes adjusted to administer a total of 1 mg/kg. Higher dose levels, longer infusion times and/or multiple infusions are also used instead of or in addition to the 1 mg/kg dose.

### Functional Assessment

All behavioral assessments are conducted without any restraint of the forelimb. All animals are trained on the performance tasks prior to the MCAO to establish their baseline. Rats with performance below the cutoff point during baseline measurement will be excluded. Testing includes:
3.4.1 7-point neuro severity score (NSS)
3.4.2 Limb placing
3.4.3 Cylinder test

Assessments are conducted 1, 4, 7, 14 and 28 days after injury.

T2 MRI imaging is used to assess lesion volume, edema and blood-brain-barrier integrity at 1, 3, and 7 days post injury in all animals.

### Immunohistochemistry

Sections (40 μm) are processed for immunohistochemistry. BrdU staining is preceded by DNA denaturation and incorporated BrdU is detected using sheep anti-BrdU. The following antibodies for phenotyping are applied in combination with anti-BrdU: guinea-pig anti-doublecortin (DCX), mouse anti-neuronal nuclei (NeuN) Alexa Fluor-488 conjugated, rabbit anti-Iba-1 or rabbit anti-glial fibrillary acidic protein. Subsequently, sections are incubated with appropriate secondary antibodies Alexa Fluor 594 or Alexa Fluor 488 for immunofluorescent labeling. To detect cells undergoing apoptosis, sections are stained for cell death using a TdT-mediated dUTP-biotin nick-end labeling (TUNEL) assay according to the manufacturer's instructions.

### Neuroanatomical Analysis

Six pyramidal neurons per animal ipsilateral and contralateral hippocampus are selected at random for analysis. Criteria for inclusion in the analyses are that the neuron much be well impregnated, unobstructed by other dendrites, blood vessels or glial cells, and the dendritic arborizations intact and visible in the plane of the section. Dendritic arborizations and lengths are analyzed by Sholl analysis. For all analyses the slides will be coded and investigators are blind to the treatment group.

### Statistical Methods

All data, including mortality across treatment groups is analyzed using parametric or nonparametric ANOVA or similar, appropriate statistical tests with p-values adjusted for multiple comparisons. Normality and equality of variances among the groups is analyzed with normality test and Barlett test, respectively.

### Example 7—Identification of Optimal Exposure Conditions of Cortical Neurons to DMT for Maximal Neurite Outgrowth

The objective of this study was the identification of the optimal conditions of primary rat cortical neurons to DMT and ketamine for maximal neurite outgrowth.

CD rat cortical cultures were prepared from E18 embryos. The cultures were stimulated for 1 h, 6 h, 12 h, 24 h and 72 h with various DMT and 10 nM Ketamine concentrations on DIV3, followed by up to 71 h growth period without stimulation. At the end-point, on DIVE, cortical cultures formaldehyde fixed and stained by using microtubules associated protein (MAP-2) immunocytochemistry. The study end-points included evaluation of neurite outgrowth.

The following equipment and materials were used for the study: Humidified incubator (Heraeus/VWR); Dissection microscope (Nikon); Zeiss AxioVert A1 inverted microscope (Zeiss); ImageJ Image v1.48e analysis software (NIH).

The following reagents and solutions were used for the study: Thermo Scientific™ Nunc™ Cell-Culture Treated Multidishes, 24 well/plates. The replacement medium was 1× B27 supplement (Life Technologies), 1% penicillin-streptomycin, 0.5 mM glutamine and 12.5 μM glutamate in Neurobasal (No DMSO addition). This replacement medium solution was administered to cells the cells after the stimulation was performed and cells were allowed to grow for another 71 h in the fresh replacement medium.

Rats (Sprague Dawley) were euthanized with CO2 and cervical dislocation. Abdomen was sprayed throughout with EtOH and skin and other layers were cut open. Hysterectomy was done by finding the ovaries and cutting the horns of the uterus intact and finally cutting the vagina. The whole uterus, pups still inside, was placed in ice-cold HBSS-A buffer and delivered immediately to biomarker on ice.

The cortical mixed cultures are prepared from E18 CD rat embryos (CRL, Germany). The cortices were dissected out and the tissue was cut to small pieces. The cells were separated by 15-min incubation with DNase and papain. The cells were collected by centrifugation (1500 rpm, 5 min). The tissue was triturated with a pipette and the cells were plated (25 000 cells in 500 μl medium) on poly-D-lysine coated 24 wells, in 10% heat-inactivated fetal bovine serum (FBS; Life Technologies), 1% penicillin-streptomycin (Life Technologies), and 0.5 mM glutamine (Life Technologies) in Neurobasal medium (Life Technologies). After 16-24 from plating, the cell culture medium was changed for replacement media consisting of 1× B27 supplement (Life Technologies), 1% penicillin-streptomycin, 0.5 mM glutamine and 12.5 μM glutamate in Neurobasal.

The study experimental design was as follows (Table 1):

The study groups were Vehicle, DMT, Ketamine, as follows:
1. Replacement medium for DMT/Ketamine exposure for 1 h+remove exposure+add replacement medium for 71 h follow up period
2. Replacement medium for DMT/Ketamine exposure for 6 h+remove exposure+add replacement medium for 66 h follow up period
3. Replacement medium for DMT/Ketamine exposure for 12 h+remove exposure+add replacement medium for 60 h follow up period
4. Replacement medium for DMT/Ketamine exposure for 24 h+remove exposure+add replacement medium for 48 h follow up period
5. Replacement medium for DMT/ketamine exposure for 72 h
6. DMT (3 μM) exposure for 1 h+remove exposure+add replacement medium for 71 h follow up period
7. DMT (3 μM) exposure for 6 h+remove exposure+add replacement medium for 66 h follow up period
8. DMT (3 μM) exposure for 12 h+remove exposure+add replacement medium for 60 h follow up period
9. DMT (3 μM) exposure for 24 h+remove exposure+add replacement medium for 48 h follow up period
10. DMT (3 μM) exposure for 72 h
11. DMT (0.3 μM) exposure for 1 h+remove exposure+add replacement medium for 71 h follow up period
12. DMT (0.3 μM) exposure for 6 h+remove exposure+add replacement medium for 66 h follow up period
13. DMT (0.3 μM) exposure for 12 h+remove exposure+add replacement medium for 60 h follow up period
14. DMT (0.3 μM) exposure for 24 h+remove exposure+add replacement medium for 48 h follow up period
15. DMT (0.3 μM) exposure for 72 h
16. DMT (30 nM) exposure for 1 h+remove exposure+add replacement medium for 71 h follow up period
17. DMT (30 nM) exposure for 6 h+remove exposure+add replacement medium for 66 h follow up period
18. DMT (30 nM) exposure for 12 h+remove exposure+add replacement medium for 60 h follow up period
19. DMT (30 nM) exposure for 24 h+remove exposure+add replacement medium for 48 h follow up period
20. DMT (30 nM) exposure for 72 h
21. DMT (3 nM) exposure for 1 h+remove exposure+add replacement medium for 71 h follow up period
22. DMT (3 nM) exposure for 6 h+remove exposure+add replacement medium for 66 h follow up period
23. DMT (3 nM) exposure for 12 h+remove exposure+add replacement medium for 60 h follow up period
24. DMT (3 nM) exposure for 24 h+remove exposure+add replacement medium for 48 h follow up period
25. DMT (3 nM) exposure for 72 h
26. DMT (0.3 nM) exposure for 1 h+remove exposure+add replacement medium for 71 h follow up period
27. DMT (0.3 nM) exposure for 6 h+remove exposure+add replacement medium for 66 h follow up period
28. DMT (0.3 nM) exposure for 12 h+remove exposure+add replacement medium for 60 h follow up period
29. DMT (0.3 nM) exposure for 24 h+remove exposure+add replacement medium for 48 h follow up period
30. DMT (0.3 nM) exposure for 72 h
31. Ketamine 0.01 μM exposure for 1 h+remove exposure+add replacement medium for 71 h follow up period
32. Ketamine 0.01 μM exposure for 6 h+remove exposure+add replacement medium for 66 h follow up period
33. Ketamine 0.01 μM exposure for 12 h+remove exposure+add replacement medium for 60 h follow up period
34. Ketamine 0.01 μM exposure for 24 h+remove exposure+add replacement medium for 48 h follow up period
35. Ketamine 0.01 μM exposure for 72 h
   The same exposures for the same time point were placed in the same plate if possible. Only the middle wells were used

Neurite outgrowth was quantified by immunostaining the neuronal microtubules with the specific antibody MAP-2. Briefly, after removing the culture medium the cultures were fixed with 4% formaldehyde solution in 1×PBS for 30 min and then washed twice with PBS. The cells are then washed twice with 1×PBS, permeabilized, and blocked against non-specific binding by a 30 min incubation with blocking buffer containing 1% bovine serum albumin and 0.3% Triton X-100 in PBS.

The cells were incubated with the primary antibody, rabbit anti-MAP-2 (Millipore, catalog #AB5622, 1:1,000), for 24 h at RT, washed with 1×PBS and then incubated for 2 h with a secondary antibody, goat anti-rabbit IgG conjugated to Alexa Fluor568 (Life Technologies catalog #A11036, 1:200) at RT. The cellular nuclei were stained by adding DAPI (2-(4-Amidinophenyl)-6-indolecarbamidine dihydrochloride) (Sigma, catalog #D8417). DAPI is cell permeable fluorescent probe for DNA. After removing 0.01M PBS from the cultures DAPI 0.1 μg/mL in 0.01M PBS was added for 2 minutes. DAPI was removed and the cells were washed three times with 1×PBS.

Each well was imaged five locations using AxioVert A1 microscope (Carl Zeiss) using an LD A-Plan 20× objective (NA 00/1.0 (PS)) (Carl Zeiss). The system was coupled to an AxioCam monochrome camera that was used to capture the images. The captured images were exported as tiff files using the ZEN software (Carl Zeiss). The tiff images were used for the neurite outgrowth analysis using the image analysis software FIJI using SNT plugin. Briefly, selected neurons were manually traced for the analysis of the total length and count of the different processes and branches from each analyzed neuron.

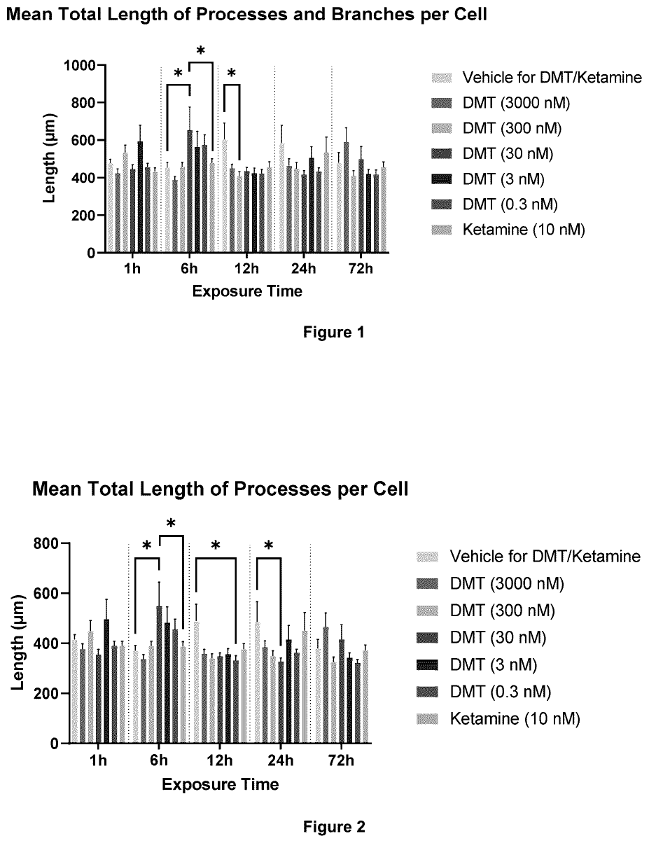

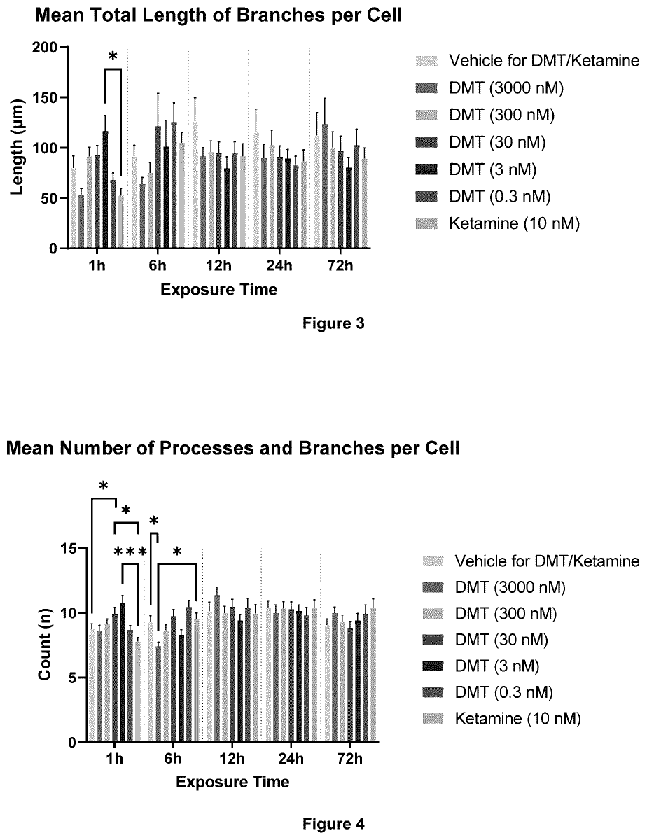

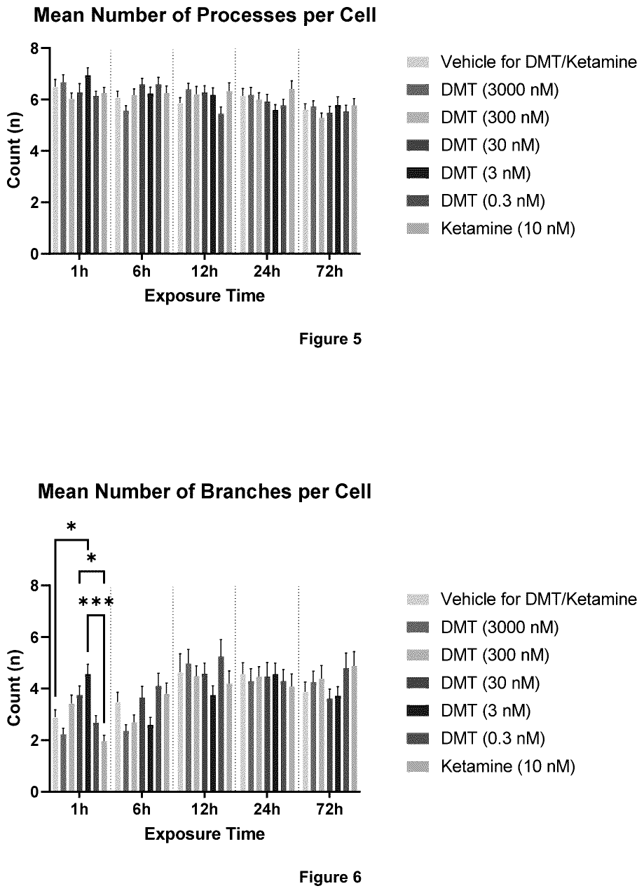

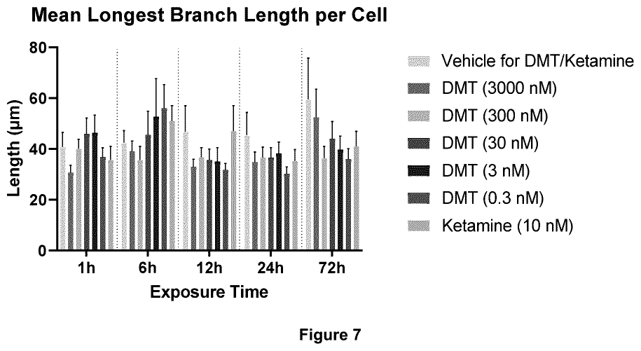

Different parameters were evaluated from the neurite outgrowth analysis: mean total length of processes and branches per cell (FIG. 1), mean total length of processes per cell (FIG. 2), mean total length of branches per cell (FIG. 3), mean number of processes and branches per cell (FIG. 4), mean number of processes per cell (FIG. 5), and mean number of branches per cell (FIG. 6). Also the length of the longest branch per cell was determined (FIG. 7). Total of 36 neurons (=12 neurons*3 wells) per treatment group was quantified for neurite outgrowth analysis. The detailed statistical comparisons and results are presented in FIGS. 1-7.

DMT stimulation showed beneficial effects on the neurite outgrowth of rat cortical neurons. Six hour stimulation with 30 nM DMT significantly increased the total length of processes and branches (FIG. 1) and the increase in the total length was more specifically present in the processes (FIG. 2). The number of processes and branches was increased with one hour stimulation with 30 nM DMT (FIG. 4). One hour stimulation with 3 nM DMT significantly increased the number of branches (FIG. 6). The reference test article, 10 nM Ketamine, did not show any significant beneficial effects in the neurite outgrowth (FIGS. 1-7).

FIG. 1 shows the Mean Total Length of Processes and Branches per Cell. Data are presented as mean+SEM. Total of 36 neurons (=12 neurons*3 wells) per treatment group were analyzed. Statistical significances: * p<0.05, DMT (30 nM) vs. Vehicle and DMT (30 nM) vs. Ketamine (10 nM) 6 h timepoint; DMT (300 nM) vs. Vehicle 12 h timepoint, (Two-way ANOVA, Dunnett's multiple comparisons test). In each Figure, the compound and dosage tested are presented in the order that they are listed to the right of the graph.

FIG. 2 shows the Mean Total Length of Processes per Cell. Data are presented as mean+SEM. Total of 36 neurons (=12 neurons*3 wells) per treatment group were analyzed. Statistical significances: * p<0.05, DMT (30 nM) vs. Vehicle and DMT (30 nM) vs. Ketamine (10 nM) 6h timepoint; DMT (0.3 nM) vs. Vehicle 12 h timepoint; DMT (30 nM) vs. Vehicle 24 h timepoint, (Two-way ANOVA, Dunnett's multiple comparisons test).

FIG. 3 shows the Mean Total Length of Branches per Cell. Data are presented as mean+SEM. Total of 36 neurons (=12 neurons*3 wells) per treatment group were analyzed. Statistical significances: * p<0.05, DMT (3 nM) vs. Ketamine (10 nM) 1h timepoint (Two-way ANOVA, Dunnett's multiple comparisons test).

FIG. 4 shows the Mean Number of Processes and Branches per Cell. Data are presented as mean+SEM. Total of 36 neurons (=12 neurons*3 wells) per treatment group were analyzed. Statistical significances: * p<0.05, DMT (30 nM) vs. Vehicle and DMT (30 nM) vs. Ketamine (10 nM), *** p<0.001, DMT (3 nM) vs. Ketamine (10 nM) 1h timepoint; DMT (3000 nM) vs. Vehicle and DMT (3000 nM) vs. Ketamine (10 nM) 6h timepoint, (Two-way ANOVA, Dunnett's multiple comparisons test).

FIG. 5 shows the Mean Number of Processes per Cell. Data are presented as mean+SEM. Total of 36 neurons (=12 neurons*3 wells) per treatment group were analyzed. No statistical significances were observed when comparing treatment groups p>0.05 (Two-way ANOVA).

FIG. 6 shows the Mean Number of Branches per Cell. Data are presented as mean+SEM. Total of 36 neurons (=12 neurons*3 wells) per treatment group were analyzed. Statistical significances: * p<0.05, DMT (3 nM) vs. Vehicle and DMT (30 nM) vs. Ketamine (10 nM) 1 h timepoint; *** p<0.001, DMT (3 nM) vs. Ketamine (10 nM) 1 h timepoint (Two-way ANOVA, Dunnett's multiple comparisons test).

FIG. 7 shows the Mean Longest Branch Length per Cell. Data are presented as mean+SEM. Total of 36 neurons (=12 neurons*3 wells) per treatment group were analyzed. No statistical significances were observed when comparing treatment groups p>0.05 (Two-way ANOVA).

Graphpad statistical software Six hour stimulation with 30 nM DMT significantly increased the total length of processes and branches and the increase in the length was more specifically present in the processes. The number of processes and branches was increased with one hour stimulation with 30 nM DMT. One hour stimulation with 3 nM DMT significantly increased the number of branches. The reference test article, 10 nM Ketamine, did not show any significant beneficial effects in the neurite outgrowth.

### Dosing DMT and Nicotinate or Pamoate Salts for the Treatment of Neuronal Injury

DMT is known to possess hallucinatory effects, in addition to its positive effects on neuroplasticity. It is also known that neuroplasticity can be achieved at lower doses below doses which are predicted to be hallucinogenic (Ly C, Greb A C, Cameron L P, Ori-Mckenney K M, Gray J A, Olson Correspondence D E. Psychedelics Promote Structural and Functional Neural Plasticity. Cell Rep. 2018; 23:3170-3182). Hallucinations are an adverse event that could prevent widespread clinical use, especially in patients suffering from stroke, multiple sclerosis, Parkinson's disease, and traumatic brain injury (Lipton S A, Failures and Successes of NMDA Receptor Antagonists: Molecular Basis for the Use of Open-Channel Blockers like Memantine in the Treatment of Acute and Chronic Neurologic Insults. NeuroRx. 2004; 1(1):101-110). Therefore a sub-hallucinogenic dose of DMT that still retains a positive effect on neuroplasticity is of clinical benefit.

Preferred target blood levels of DMT and nicotinate or pamoate DMT that are below 250 ng/mL, preferably below 150 ng/mL, preferably below 40 ng/mL, preferably below 30 ng/mL, preferably below 20 ng/mL, preferably below 10 ng/mL, for the treatment of stroke, multiple sclerosis, Parkinson's disease, and traumatic brain injury with absent or minimal hallucinatory effects.

### Duration

The duration of drug exposure can be important variable for triggering neuroplasticity (Ly C, Greb A C, Vargas M V., et al. Transient Stimulation with Psychoplastogens Is Sufficient to Initiate Neuronal Growth. ACS Pharmacol Transl Sci. September 2020). DMT is rapidly cleared from the body (half-life 15 minutes) and is not orally available, therefore in order to achieve a sustained target concentration for a defined time, the product will be continuously intravenously infused. Although a short exposure can trigger improvements in neuroplasticity, longer exposures are better.

Therefore an infusion duration of DMT, pamoate DMT, or nicotinate DMT of at least 15 minutes and up to 24 hours, preferable a six-hour infusion window is preferred.

Use with Antihypertensives

DMT can have blood-pressure elevating effects. Strassman et al found that the effects on blood pressure and heart rate are dose dependent. The present inventors have thus achieved neuroplasticity with a dose that has minimal effect on the circulatory system. Furthermore, the hypertensive features of DMT are offset by co-administration with an anti-hypertensive therapy. This is important when treating stroke or TBI.

The anti-hypertensive therapy can be a drug selected from the group of calcium channel blockers, renin-angiotensin system inhibitors, diuretics, adrenergic receptor antagonists, aldosterone antagonists, vasodilators, Alpha-2 agonists and pharmaceutically acceptable salts thereof.

### Use of DMT and Salts Thereof in the Rehabilitative Process

Transient exposure to psychedelic agents LSD and ketamine produces neuronal growth in vitro which is not necessarily immediate, but which peaks after some time—approximately 3 days. DMT and in particular pamoate DMT or nicotinate DMT administration before therapy allows the neuroplasticity to be at its highest during therapy.

In constrained therapy, the healthy side of a stroke patient's body is constrained, forcing them to use the affected side to perform various tasks, leading to a recovery of function in the affected side. Since one is training new neural pathways, enhanced neuronal plasticity may be beneficial in this treatment regimen.

Constraint-Induced Movement Therapy (CIMT) has controlled evidence of efficacy for improving real-world paretic limb use in non-progressive physically disabling disorders (stroke, cerebral palsy)(Mark, VW, Phase II Randomized Controlled Trial of Constraint-Induced Movement Therapy in Multiple Sclerosis. Part 1: Effects on Real-World Function, Neurorehabilitation and Neural Repair, Vol 32, Issue 3, 2018).

In addition to the treatment of stroke patients, it is appreciated that the compositions, methods and uses of the present invention can be used for the treatment or prevention of Parkinson's disease, dyskinesias, dystonias, Tourette's disease, iatrogenic and non-iatrogenic psychoses and hallucinoses, mood and anxiety disorders, sleep disorder, autism spectrum disorder, ADHD, Huntington's disease, age-related cognitive impairment, and disorders related to alcohol abuse and narcotic substance abuse.

Thus the use of DMT, and in particular pamoate or nicotinate salts of DMT are particularly useful in CIMT rehabilitation, which includes motor-skill exercises, mobility training, constraint-induced and range-of-motion therapy, and can begin as soon as 24 to 48 hours after the stroke has occurred.

Additionally, other forms of rehabilitation can be used to stimulate neuronal growth and new neuronal connections and be used with the present invention to allow for enhanced neuronal plasticity. Such methods of rehabilitation include, but are not limited to, motor skill exercises (i.e. therapy ball, therapy putty, tabletop exercises, object moving, object stacking, resistance exercises), mobility therapy (i.e. balance and coordination exercises), range of motion therapy (i.e. stretching, reaching exercises, circular and pushing movements, joint rotations), functional electrical stimulation (i.e. functional neuromuscular stimulation, electrical stimulation, TENS, neuroprothesis), robotic technology (i.e. haptic interfaces, exoskeletons, supportive assemblies), occupational therapies, speech therapy, cognitive therapy (i.e. visual/auditory memory exercises, visual/spatial processing, analytical reasoning, quantitative reasoning, meditation).

In addition to the treatment of stroke patients, the compositions, methods and uses of the present invention can be used for the treatment or prevention of other neurodegenerative diseases, such as multiple sclerosis, Parkinson's disease, and traumatic brain injury.

DMT and DMT salts can promote neurogenesis and structural and functional neural plasticity during various time periods long after the stroke has occurred.

### Example 8—Radioligand Binding Assays

Radioligand binding assays were used to evaluate the activity of the test compound(s) N,N-Dimethyltryptamine fumarate, N,N-Dimethyltryptamine pamoate, and N,N-Dimethyltryptamine nicotinate.

The assays looked at binding to 5HT2A and sigma-1 receptors. DMT is known to bind to 5HT2A and sigma-1. These receptors are highly correlated with neuronal activity, and in particular with DMT activity in the brain.

5HT2A also has a role in treating neurodegenerative diseases Alzheimers and Parkinsons. (Herth and Knudsen, Labelled Compounds and Radiopharmaceuticals, Vol. 58:7, 265-273, 15 Jun. 2015). The sigma-1 receptor enhances brain plasticity and functional recovery after experimental stroke (Ruscher et al, Brain. 2011 March; 134 (Pt3):732-46. 2011 Jan. 28).

The sigma-1 receptor (61R) is a chaperone protein at the endoplasmic reticulum (ER) that modulates calcium signaling through the IP3 receptor. The 61 receptor is a transmembrane protein concentrated in certain regions of the central nervous system (Shi et al, Frontiers in Cellular Neuroscience, 15:1-19, article 685201, September, 2021; Ryskamp, Frontiers in Cellular Neuroscience, 13:1-20, article 862, August 2019; Nguyen et al, Adv Exp Med Biol, 964:133-152, 2017).

IC50 values were determined by a non-linear, least squares regression analysis using MathIQ™ (ID Business Solutions Ltd., UK). The Ki values were calculated using the equation of Cheng and Prusoff (Cheng, Y., Prusoff, W. H., Biochem. Pharmacol. 22:3099-3108, 1973) using the observed IC50 of the tested compound, the concentration of radioligand employed in the assay, and the historical values for the KD of the ligand (obtained experimentally at Eurofins Panlabs, Inc.). The Hill coefficient (nH), defining the slope of the competitive binding curve, was calculated using MathIQ™. Significant results are displayed in the following table(s) in rank order of potency for estimated IC50 and/or Ki values.

Biochemical assay results are presented as the percent inhibition of specific binding or activity. All other results are expressed in terms of that assay's quantitation method. Primary screening in duplicate with semi-quantitative data (e.g., estimated IC50, Ki and nH) are shown. Significant responses 50% inhibition or stimulation for Biochemical assays) were found in the primary assays listed below.

TABLE 2

| | | | | | | | |
|---|---|---|---|---|---|---|---|
| Cat # | Assay Name | Species | Conc. | % Inh. | IC₅₀* | Kᵢ | n_{H} |

Compound: N,N-Dimethyltryptamine fumarate, PT #: 1257430

| | | | | | | | | | | |
|---|---|---|---|---|---|---|---|---|---|---|
| 271650 | Serotonin (5-Hydroxytryptamine) 5-HT_{2A} | human | 1 | μM | 76 | 0.25 | μM | 0.071 | μM | 0.75 |
| 299034 | Sigma 0₁ | human | 10 | μM | 50 | 10.2 | μM | 5.28 | μM | 0.76 |

Compound: N,N-Dimethyltryptamine nicotinate, PT #:

[symbol]

| | | | | | | | | | | |
|---|---|---|---|---|---|---|---|---|---|---|
| 271650 | Serotonin (5-Hydroxytryptamine) 5-HT_{2A} | human | 1 | μM | 59 | 0.60 | μM | 0.17 | μM | 0.71 |

Compound: N,N-Dimethyltryptamine pamoate, PT #: 1257431

| | | | | | | | | | | |
|---|---|---|---|---|---|---|---|---|---|---|
| 271650 | Serotonin (5-Hydroxytryptamine) 5-HT_{2A} | human | 0.3 | μM | 56 | 0.19 | μM | 0.054 | μM | 0.92 |
| 299034 | Sigma 0₁ | human | 10 | μM | 64 | 6.31 | μM | 3.25 | μM | 1.35 |

[symbol]

indicates data missing or illegible when filed

TABLE 3

| | | | | | | | | |
|---|---|---|---|---|---|---|---|---|
| Assay Name | Batch* | Spec. | Rep. | Conc. % | Inh. | IC₅₀ | Kᵢ | nH |

Compound: N,N-Dimethyltryptamine fumarate

| | | | | | | | | | |
|---|---|---|---|---|---|---|---|---|---|
| Serotonin (5-Hydroxytryptamine) | 481827 | hum | 2 | 10 | μM | 98 | 0.25 μM | 0.071 μM | 0.75 |
| | | hum | 2 | 3 | μM | 93 | | | |
| | | hum | 2 | 1 | μM | 76 | | | |
| | | hum | 2 | 0.3 | μM | 48 | | | |
| | | hum | 2 | 0.1 | μM | 26 | | | |
| | | hum | 2 | 0.03 | μM | 24 | | | |
| | | hum | 2 | 10 | nM | 15 | | | |
| | | hum | 2 | 3 | nM | 8 | | | |
| Sigma 01 | 482026 | hum | 2 | 16 | μM | 59 | 10.2 μM | 5.28 μM | 0.76 |
| | | hum | 2 | 10 | μM | 50 | | | |
| | | hum | 2 | 5 | μM | 36 | | | |
| | | hum | 2 | 1 | uM | 14 | | | |
| | | hum | 2 | 0.5 | μM | 5 | | | |
| | | hum | 2 | 0.1 | μM | 7 | | | |
| | | hum | 2 | 0.03 | μM | 15 | | | |
| | | hum | 2 | 10 | nM | 6 | | | |

Compound: N,N-Dimethyltryptamine nicotinate

| | | | | | | | | | |
|---|---|---|---|---|---|---|---|---|---|
| Serotonin (5-Hydroxytryptamine) | 481827 | hum | 2 | 10 | μM | 92 | 0.60 μM | 0.17 μM | 0.71 |
| | | hum | 2 | 3 | μM | 79 | | | |
| | | hum | 2 | 1 | μM | 59 | | | |
| Sigma 01 | | hum | 2 | 0.3 | μM | 31 | | | |
| | | hum | 2 | 0.1 | μM | 19 | | | |
| | | hum | 2 | 0.03 | μM | 17 | | | |
| | | hum | 2 | 10 | nM | 14 | | | |
| | | hum | 2 | 3 | nM | 7 | | | |
| | 482026 | hum | 2 | 16 | μM | 44 | | | |
| | | hum | 2 | 10 | μM | 44 | | | |
| | | hum | 2 | 5 | μM | 23 | | | |
| | | hum | 2 | 1 | uM | 5 | | | |
| | | hum | 2 | 0.5 | μM | −13 | | | |
| | | hum | 2 | 0.1 | μM | −3 | | | |
| | | hum | 2 | 0.03 | μM | −9 | | | |
| | | hum | 2 | 10 | nM | −6 | | | |

| | | | | | | | | |
|---|---|---|---|---|---|---|---|---|
| Assay Name | Batch* | Spec. | Rep. | Conc. % | Inh. | IC50* | Ki | nH |

Compound: N,N-Dimethyltryptamine pamoate,

| Serotonin (5-Hydroxytryptamine) | 481827 | hum | 2 | 10 | μM | 100 | 0.19 μM | 0.054 μM | 0.92 |
|---|---|---|---|---|---|---|---|---|---|
| 5-HT2A | | hum | 2 | 3 | μM | 95 | | | |
| | | hum | 2 | 1 | | 86 | | | |
| | | hum | 2 | μM | | 56 | | | |
| | | hum | 2 | 0.1 | μM | 34 | | | |
| | | hum | 2 | 0.03 | μM | 20 | | | |
| | | hum | 2 | 10 | nM | 10 | | | |
| | | hum | 2 | 3 | nM | 2 | | | |
| Sigma 01 | 482026 | hum | 2 | 16 | μM | 77 | 6.31 μM | 3.25 μM | 1.35 |
| | | hum | 2 | 10 | μM | 64 | | | |
| | | hum | 2 | 5 | μM | 45 | | | |
| | | hum | 2 | 1 | uM | 6 | | | |
| | | hum | 2 | 0.5 | μM | 1 | | | |
| | | hum | 2 | 0.1 | μM | 0 | | | |
| | | hum | 2 | 0.03 | μM | −10 | | | |
| | | hum | 2 | 10 | nM | −14 | | | |

Note:
Items meeting criteria for significance (>50% stimulation or inhibition) are highlighted.
*Batch: Represents compounds tested concurrently in the same assay(s).
hum = Human
“N.C. = Not calculated”.
All the % inhibition is more than 50, unable to calculate IC50, Ki, and nH
All the % inhibition is less than 50, unable to calculate IC50, Ki, and nH

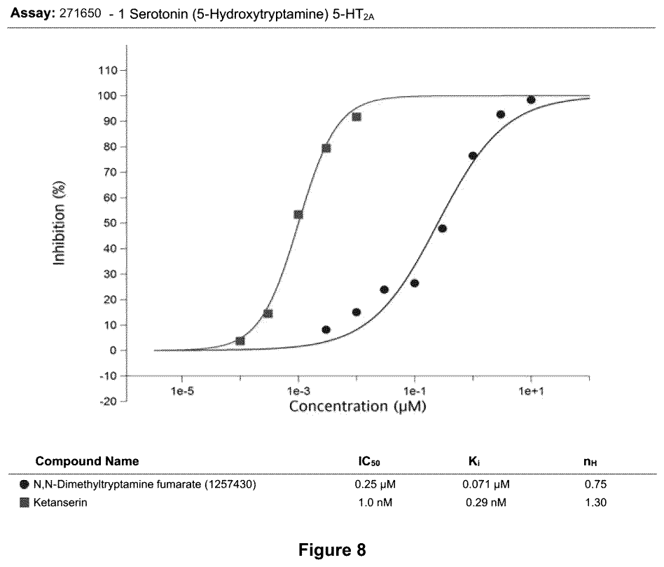

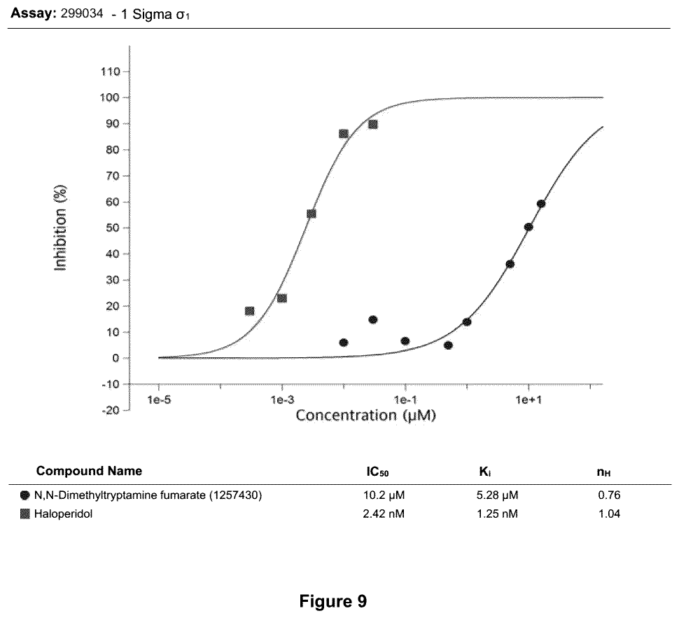

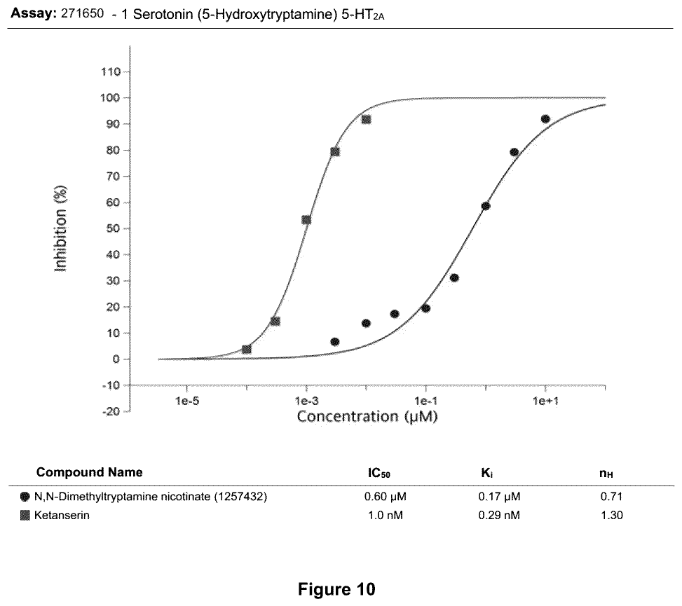

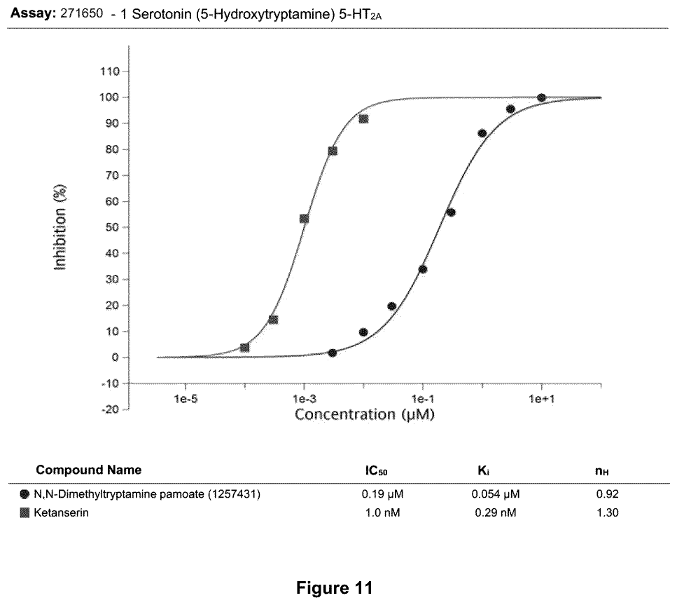

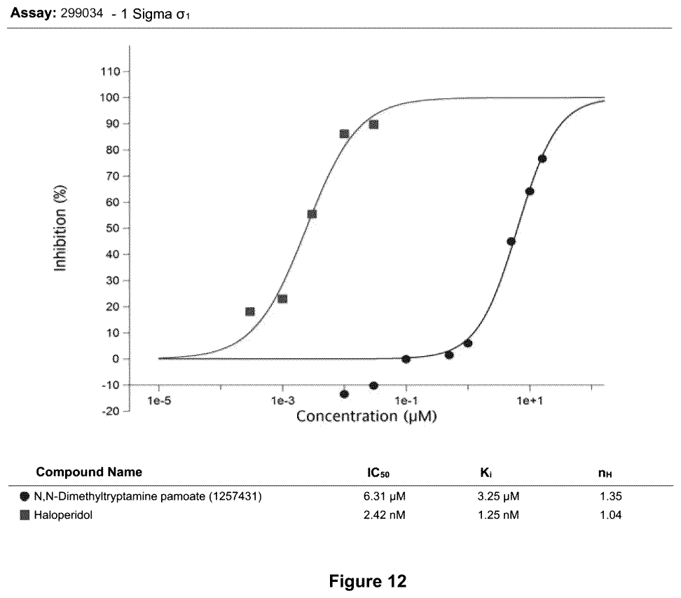

Significant response curves for the different salts are shown in FIGS. 8 to 12. FIG. 8 shows DMT fumarate's binding activity to 5HT2A receptor against a standard. Ketanserin is a well-known selective 5HT2A receptor antagonist. FIG. 9 is a line graph showing DMT fumarate's binding activity to sigma-1 receptor against a standard. Haloperidol is a well-known selective sigma-1 receptor antagonist. FIG. 10 shows DMT nicotinate's binding activity to 5HT2A receptor against the standard. FIG. 11 shows DMT pamoate's binding activity to 5HT2A receptor against the standard. FIG. 12 shows DMT pamoate's binding activity to sigma-1 receptor against the standard.

The results show DMT nicotinate (Ki=0.17 μM; FIG. 3) exhibited high affinity to the 5HT2a receptor while also showing good binding affinity to sigma-1-receptors at concentrations above 0.5 μM (Table 3).

DMT pamoate showed comparable 5HT2A binding (Kᵢ=0.054 μM; FIG. 4) to DMT fumarate (Kᵢ=0.071 μM; FIG. 1). DMT pamoate showed improved 5HT2A binding compared to DMT fumarate at concentrations above 0.1 μM (Table 3). DMT pamoate also showed comparable binding (Ki=3.25 μM; FIG. 5) to sigma-1 receptors compared to DMT fumarate (Ki=5.28 μM; FIG. 2). DMT pamoate also is showed better affinity to sigma-1 receptors compared to DMT fumarate at concentrations above 1 μM (Table 3).

Thus, DMT nicotinate displayed clear binding potency and good affinity to the receptors 5HT2a and sigma-1. DMT pamoate has better 5HT2A and sigma-1 receptor affinity over DMT fumarate at higher concentrations. Using the evidence provided herein and as these receptors are highly correlated with neuronal activity, and in particular with DMT activity in the brain, these DMT salts can provide improvements over known salts of DMT.

1. A compound comprising pamoate DMT or nicotinate DMT.

2. A method of treating stroke, multiple sclerosis, Parkinson's disease, and traumatic brain injury, comprising administering the compound of claim 1.

3-4. (canceled)

5. The method of claim 2, or the use of a pharmaceutically acceptable form of DMT for the treatment of haemorrhagic stroke comprising administration commencing prior to a diagnosis of ischemic or haemorrhagic stroke.

6. The method of claim 5, wherein said diagnosis is by CT scan.

7. The method of claim 6, or the use of DMT for the treatment of haemorrhagic stroke.

8. The method of claim 2, or the use of a pharmaceutically acceptable form of DMT for the treatment of stroke, multiple sclerosis, Parkinson's disease, and traumatic brain injury, comprising administration in combination with rehabilitative therapy such as constrained exercise, motor skill exercises (i.e. therapy ball, therapy putty, tabletop exercises, object moving, object stacking, resistance exercises), mobility therapy (i.e. balance and coordination exercises), range of motion therapy (i.e. stretching, reaching exercises, circular and pushing movements, joint rotations), functional electrical stimulation (i.e. functional neuromuscular stimulation, electrical stimulation, TENS, neuroprothesis), robotic technology (i.e. haptic interfaces, exoskeletons, supportive assemblies), occupational therapies, speech therapy, cognitive therapy (i.e. visual/auditory memory exercises, visual/spatial processing, analytical reasoning, quantitative reasoning, meditation).

9. (canceled)

10. The method of claim 8, comprising administration at least 2 days before constrained exercise.

11-12. (canceled)

13. The method of claim 2 or the use of a pharmaceutically acceptable form of DMT for the treatment of stroke, multiple sclerosis, Parkinson's disease, and traumatic brain injury, comprising administration at a rate of about 0.001 to 50 mg DMT/kg patient bodyweight/hour.

14. The method of claim 13, comprising administration at a rate to of about 0.005 to 20 mg DMT/kg patient bodyweight/hour.

15. The method of claim 14, comprising administration at a rate of about 0.01 to 5 mg DMT/kg patient bodyweight/hour.

16. The method of claim 15, comprising administration at a rate of about 0.5 mg DMT/kg patient bodyweight/hour.

17. The method of claim 2, or the use of a pharmaceutically acceptable form of DMT for the treatment of stroke, multiple sclerosis, Parkinson's disease, and traumatic brain injury, comprising administration at a rate to provide a serum level of about 0.05 to 250 ng/ml.

18. The method of claim 17, comprising administration at a rate to provide a serum level of about 0.1 to 150 ng/ml.

19. The method of claim 18, comprising administration at a rate to provide a serum level of about 1.0 to 50 ng/ml.

20. The method of claim 19, comprising administration at a rate to provide a serum level of about 25 ng/ml.

21. The method of claim 2, comprising administration for a duration of about 15 minutes to 24 hours.

22. The method of claim 21, comprising administration for a duration of about 1 hour to 18 hours.

23. The method of claim 22, comprising administration for a duration of about 2 hours to 12 hours.

24. The method of claim 23, comprising administration for a duration of about 6 hours.

25. The method of claim 2, or the use of a pharmaceutically acceptable form of DMT for the treatment of stroke or TBI, further comprising administration with an antihypertensive.

26. A device for the method of claim 2, comprising an intravenous pump, said device containing a pharmaceutically acceptable form of DMT.

27-28. (canceled)

29. The device of claim 26, said device further comprising a locking system to prevent access to said compound or said DMT above the dose of 50 mg/kg patient body weight per hour.

30. The device of claim 29, wherein said locking system locks a container of the compound.

31. The device of claim 29 or 30, wherein said locking system locks adjustment of the rate of administration of the compound.
